(19) United States
(12) Patent Application Publication
(10) Pub. No.: US 2025/0073391 A1
(43) Pub. Date: Mar. 6, 2025

(54) FIXTURE FOR TESTING SYSTEM

(71) Applicant: Regeneron Pharmaceuticals, Inc., Tarrytown, NY (US)

(72) Inventors: Christopher HUNTER, Castleton-on-Hudson, NY (US); Connor BLOCK, Somerville, MA (US)

(73) Assignee: Regeneron Pharmaceuticals, Inc., Tarrytown, NY (US)

(21) Appl. No.: 18/820,557

(22) Filed: Aug. 30, 2024

Related U.S. Application Data
(60) Provisional application No. 63/580,153, filed on Sep. 1, 2023.

(51) Int. Cl. A61M 5/20

(52) U.S. Cl. CPC A61M 5/20; A61M 2005/206

(57) ABSTRACT

A fixture for an automatic needle injection is disclosed, wherein the fixture comprises a housing including a chamber, the housing including a first plurality of walls, a second plurality of walls, and a third plurality of walls, a holder, and a membrane; wherein the membrane is contained in the holder, the holder is secured within a portion of the housing, and the holder is configured to allow a portion of a needle of a syringe to pass through a portion of the holder and into the chamber of the housing.

### CROSS-REFERENCE TO RELATED APPLICATIONS

This application claims the benefit of priority from U.S. Provisional Patent Application No. 63/580,153 filed Sep. 1, 2023, which is incorporated by reference herein.

### TECHNICAL FIELD

Embodiments of this disclosure are directed to a fixture for a testing system, for example, a fixture for holding a membrane in a test system, and uses thereof.

### INTRODUCTION

Mechanical test systems, such as tensile and compression test systems, are used in laboratory settings to perform real time analyses. Examples of such devices are the Instron® Low Force Universal Testing Systems and Instron® High Force Universal Testing Systems (Instron®, www.instron.com/en-us/products/testing-systems/universal-testing-systems, incorporated by reference herein) and ZwickRoell Universal Testing Machines (ZwickRoell, https://www.zwickroell.com/products/static-materials-testing-machines/universal-testing-machines-for-static-applications/#navigation-240, incorporated by reference herein). Such mechanical testing systems are used to evaluate drug delivery devices, e.g., prefilled syringes and autoinjectors, against testing standards, e.g., standards established by the International Organization for Standardization. Such standards address the design and functional properties of syringes and ensure that such syringes operate as intended. Various tests are performed to evaluate the components of the autoinjector, e.g., the needle shield, plunger/stopper, flange, needle, luer/cone, adapter/collar, and tip/cap. To ensure proper testing, various components may be used with the mechanical test systems.

### SUMMARY OF THE DISCLOSURE

The present disclosure describes a fixture for an automatic needle injection system, the fixture comprising a housing including a chamber, the housing including a first plurality of walls, a second plurality of walls, and a third plurality of walls, a holder, and a membrane; wherein the membrane is contained in the holder, the holder is secured within a portion of the housing, and the holder is configured to allow a portion of a needle of a syringe to pass through a portion of the holder and into the chamber of the housing.

Various embodiments of the device may include one or more of the following aspects. The fixture may be configured to be secured to a bottom portion of the automatic needle injection system, wherein the syringe and fixture are coaxially aligned with the automatic needle injection system. The holder may include a first portion, a second portion, and a plurality of cutouts, and the membrane is secured in the holder between the first portion and the second portion. The first portion may include a first plurality of cutouts and the second portion may include a second plurality of cutouts, the first plurality of cutouts aligning with the second plurality of cutouts. The plurality of cutouts on the holder may designate puncture sites for receiving the needle. The holder may be configured to rotate within the housing. The membrane may have a thickness ranging from about 0.1 mm to about 1.0 mm. The holder may have a height ranging from about 1 mm to about 10 mm. The fixture may further comprise a container for receiving a sample from the syringe. The holder may be a cassette and the membrane may be configured to be fed through the cassette.

The present disclosure also describes a fixture for an automatic needle injection system, the fixture comprising a housing including a chamber, a holder including a first portion, a second portion, and a plurality of cutouts, and a membrane, wherein the membrane is secured between the first portion and the second portion of the holder; wherein the holder is secured within a portion of the housing and the holder is configured to allow a portion of a needle of a syringe to pass through a portion of the holder and into the chamber of the housing.

Various embodiments of the fixture may include one or more of the following aspects. The holder may be configured to rotate within the housing. The holder may have a height ranging from about 1 mm to about 10 mm. The fixture may further comprise a container for receiving a sample from the syringe. The plurality of cutouts on the holder may designate puncture sites for receiving the needle.

The present disclosure also describes a method for testing a syringe, the method comprising securing a fixture to an automatic needle injection system, the fixture comprising a housing including a chamber; a holder; and a membrane, wherein the membrane is contained in the holder, the holder is secured within a portion of the housing, and the holder is configured to allow a needle of the syringe to pass through the holder and into the chamber of the housing, inserting a syringe into the automatic needle injection system, and programing the automatic needle injection system such that a needle of the syringe punctures the membrane.

Various embodiments of the method may include one or more of the following aspects. Securing the fixture to a base portion of the test system. The test system may be adjusted such that an unused portion of the membrane is punctured for each test run. The needle may extend through a portion of the housing, a portion of the holder, and into the chamber. The membrane may be punctured at least 10 times.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect or embodiment thereof, nor is it limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

FIG. 1 is a side view of a fixture, according to embodiments of the present disclosure.

FIG. 2A is a perspective view of the fixture and FIG. 2B is a perspective view of a membrane holder, according to an embodiment of the present disclosure.

FIG. 3 is a side view of the fixture, according to an embodiment of the present disclosure.

FIG. 4A is a front view of the fixture utilized in a test system and FIG. 4B is a perspective view of the fixture utilized in a test system, according to embodiments of the present disclosure.

FIG. 5 is a top view of the fixture, according to an embodiment of the present disclosure.

FIG. 6 is a side view of the fixture, according to an embodiment of the present disclosure.

FIG. 7 is a detailed view of a portion of the fixture in FIG. 6.

FIG. 8 is a detailed view of a portion of the fixture in FIG. 6.

As used herein, the terms “comprises,” “comprising,” “includes,” “including,” or

any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. In the discussion that follows, relative terms such as “about,” “substantially,” “approximately,” etc. are used to indicate a possible variation of a numerical range in a stated numeric value, as will be designated below.

As described above, various tests may be performed to evaluate needle-based injection systems or automatic injection needle systems, e.g., syringes and autoinjectors, according to standards, e.g., standards established by the International Organization for Standardization, or more specifically, ISO 11608. Components of the autoinjectors that may be evaluated, include, e.g., the prefilled syringe or cartridges, the needle shield, plunger/stopper, flange, needle, luer/cone, adapter/collar, and/or tip/cap. The needle tip or bevel of the needle on a syringe must adhere to strict standards and specifications and may be evaluated by a needle penetration test. Analyses performed during the needle penetration test may be used to characterize the sharpness of the needle, the frictional force along the shaft of the needle, and the effectiveness of the needle tip geometry. These characteristics may impact the comfort level to the patient and the trauma caused to the injection site. In other words, the higher the force required for penetration, the higher the level of discomfort. Any reference in the disclosure to a syringe, may also be applicable to autoinjectors and other needle-based injection systems.

Updates to ISO 11608 now require testing of needle-based injections systems, e.g., syringes and autoinjectors, to ensure the needle penetrates an injection site such that the dose/sample present in the syringe is delivered at a distance of about 4 mm below the surface of the injection site. Such testing requires piercing a material that mimics human skin, e.g., a membrane comprising polyurethane and/or plastic. Such testing will be referred to in this disclosure as a puncture test or puncture testing. Since the membrane is flexible, it must be configured in the test system such that it is taut. If the membrane is not held tightly, it will fold inwards on itself, preventing the syringe needle from puncturing the membrane. When the membrane is not held in a taut position, the membrane could also sag, thereby failing to simulate a typical injection event. If the syringe needle pierces a portion of the membrane that may be sagging, measurements of interest, e.g., dose accuracy or expelled volume of a medicament at a given distance (e.g., greater than about 4 mm) from the injection site, i.e., past the injection site of the membrane, or a depth of the injection, may be inaccurate. In addition, the membrane cannot be punctured in the same place repeatedly. Piercing a portion of the membrane more than once may result in skewed measurements. The punctures should also be made far enough away from prior punctures. Too many punctures in an area of the membrane may cause the membrane to weaken or droop, preventing a proper puncture and/or a desirable penetration level/result. In order to properly repeat the puncture test, the membrane may be rotated or moved such that a new section of the membrane may be used for each test run. There are manual puncture testing components that allow the operator to re-clamp new material, e.g., membrane, for puncture, but such components are inefficient and unsuitable for use on fully automated test systems, e.g., the Instron® and ZwickRoell testing apparatus. In addition, any components used with a test system must be configured such that a vision system or viewing device, e.g., a camera, may be used to capture images and data from the puncture test. Data obtained from the viewing device may include, e.g., the length of the syringe needle that punctures the membrane prior to injection and the flow time of the sample through the syringe and/or syringe needle. To ensure proper and efficient puncture testing, fixtures configured for use with automated or semi-automated (manual movement of puncture apparatus) test systems are desired.

Although the present disclosure references the Instron® and ZwickRoell test systems, those of ordinary skill in the art will readily recognize that the concepts of the present disclosure may be utilized with any suitable or comparable testing mechanism or machine (e.g., ADMET, MECMESIN, TEST RESOURCES, etc.). Moreover, although the present disclosure makes reference to testing forces applied to a syringe, those of ordinary skill in the art will readily recognize that reference to a syringe is only exemplary, and that the concepts of the present disclosure may be used in conjunction with any suitable drug delivery device, including, but not limited to, autoinjectors and syringes (both plastic and glass, including pre-filled syringes).

Accordingly, the present disclosure is directed to various embodiments of a testing fixture for use with a syringe in an automated test system (e.g., mechanical test systems, such as, e.g., an Instron® test device or ZwickRoell test device). In some examples, the syringe is a precision dose delivery system described in WO 2019/118588 and WO 2020/247686 and US Design application Ser. Nos. 29/760,798, 29/760,796, and 29/868,089, which are herein incorporated by reference in their entireties.

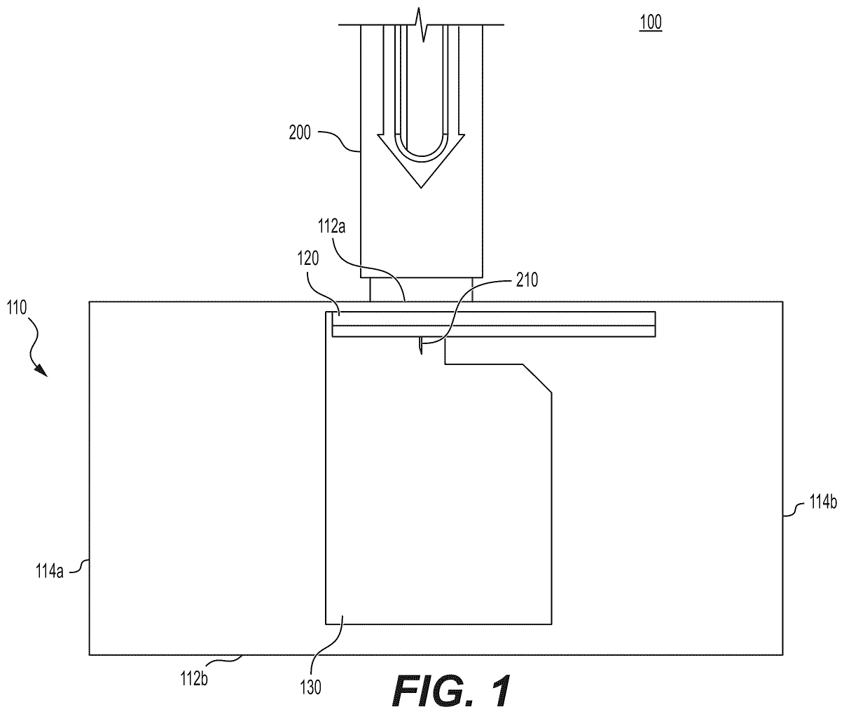

Embodiments of the present disclosure relate to a testing fixture, and, in particular, a testing fixture for holding a membrane in an automated test system (e.g., mechanical test systems, such as, e.g., an Instron® test device or ZwickRoell test device). FIG. 1 shows a side view of a fixture 100 for a test system. Fixture 100 may be formed of any suitable material with sufficient weight and components to aid in the stability of the fixture, and/or with any characteristics suitable for use in a laboratory setting and with a test system. For example, fixture 100 may be made from a plastic material and/or other chemically resistant materials. In some embodiments, fixture 100 may be made of metal machined to a desired configuration. Fixture 100 may be fabricated by any suitable method, including, but not limited to, additive manufacturing and stereolithography (SLA) methods.

Fixture 100 may include a housing 110 and a membrane holder 120. Housing 110 may include a chamber 130. When fixture 100 is utilized in a test system, a syringe 200 may abut a surface of housing 110 such that during a puncture test, a syringe needle 210 extends through housing 110 and membrane holder 120. The puncture test will be discussed in further detail throughout the disclosure.

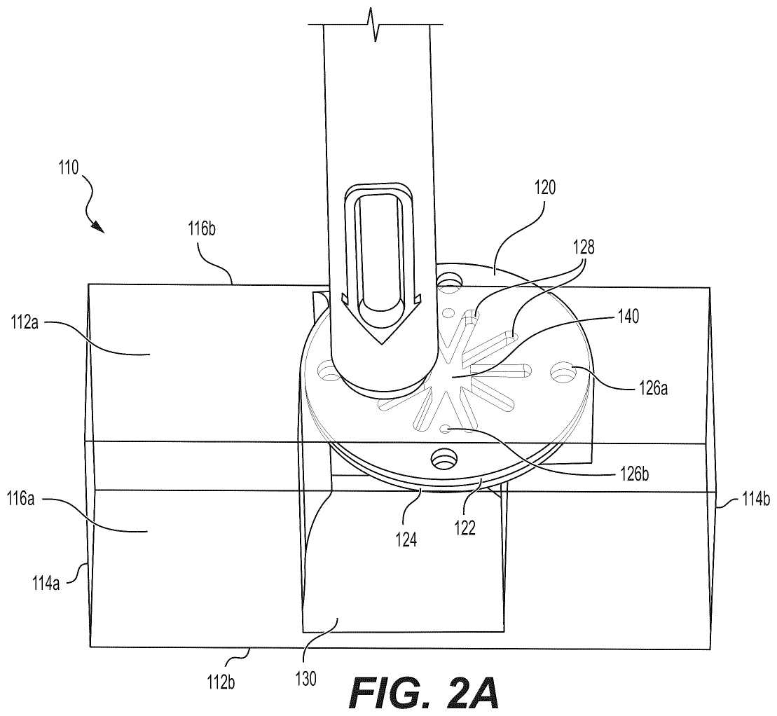

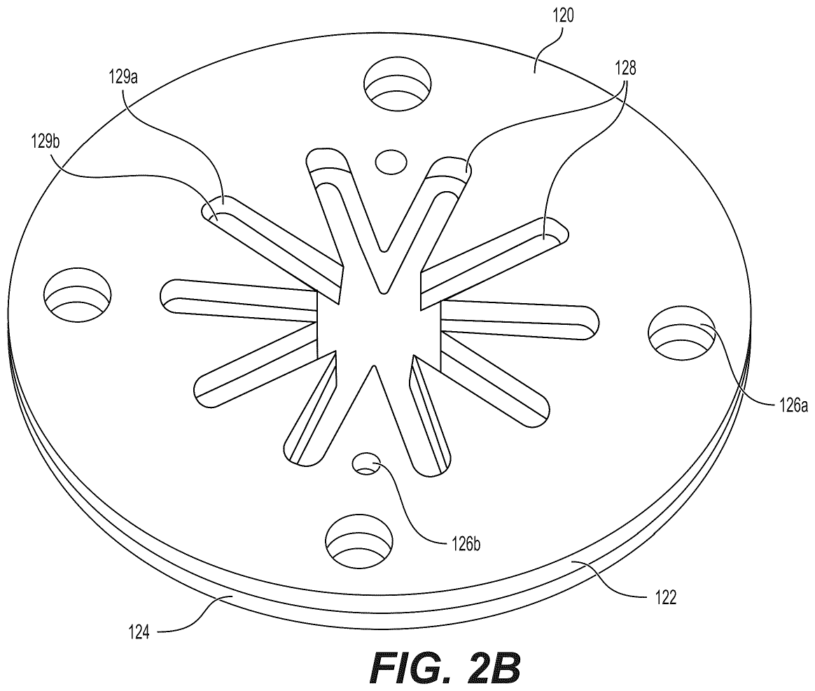

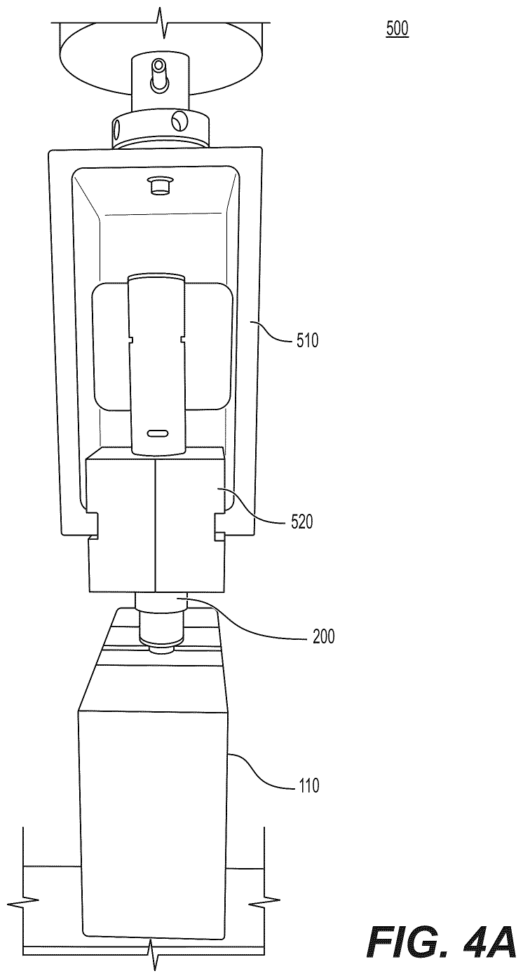

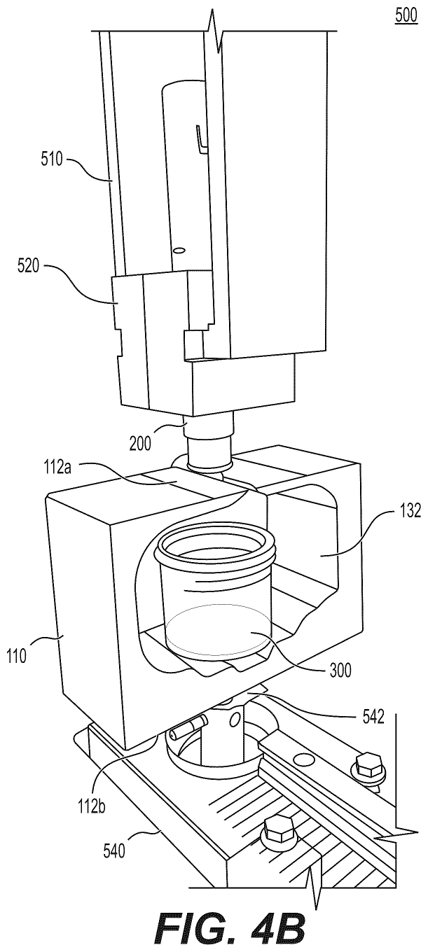

Housing 110 may include a first plurality of walls 112a, 112b, a second plurality of walls 114a, 114b, and a third plurality of walls 116a, 116b (FIGS. 1 and 2A). In some configurations, one or more of the plurality of walls may be solid. In other configurations, one of the first plurality of walls 112a, 112b may be configured to include an opening to receive a portion of syringe 200 and/or syringe needle 210 (FIGS. 4A and 4B). Referring to FIGS. 4A and 4B, one of first plurality of walls 112a, 112b, may include a slot 118 for receiving syringe needle 210.

The dimensions of housing 110 may be adjustable depending on the device, e.g., syringe or autoinjector, that is being tested. For example, dimensions of slot 118 may be adjusted to prevent the device of interest from slipping through slot 118 and into housing 110, while ensuring there is enough clearance to prevent accidental engagement between the syringe needle 210 and sample membrane 140. The measurements for housing 110 provided below are exemplary.

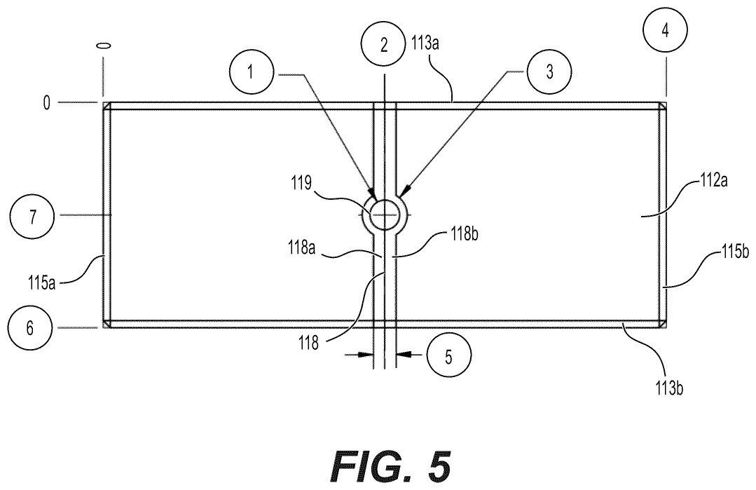

Referring to FIG. 5, a width of housing 110 represented by Measurement 4, may range from about 75 mm to about 175 mm. For example, a length of housing 110 may range from about 75 mm to about 160 mm, about 75 mm to about 145 mm, about 75 mm to about 130 mm, about 90 mm to about 175 mm, about 105 mm to about 175 mm, about 120 mm to about 175 mm, about 100 mm to about 150 mm, about 110 mm, to about 140 mm, or about 120 mm to about 130 mm. In some examples, a length of housing 110, may be about 75 mm, about 80 mm, about 85 mm, about 90 mm, about 95 mm, about 100 mm, about 105 mm, about 110 mm, about 115 mm, about 120 mm, about 125 mm, about 130 mm, about 135 mm, about 140 mm, about 145 mm, about 150 mm, about 155 mm, about 160 mm, about 165 mm, about 170 mm, or about 175 mm.

A midpoint of the width of housing 110 is represented by Measurement 2. Measurement 2 is in reference to the 0 measurement, which represents the datum or start of the dimension. Measurement 2 represents the distance from the 0 measurement. For example, a midpoint of the width of housing 110 may range from about 40 mm to about 70 mm from the 0 measurement. For example, a midpoint of the width of housing 110 may range from about 40 mm to about 65 mm, about 45 mm to about 75 mm, about 45 mm to about 65 mm, about 50 mm to about 70 mm, about 50 mm to about 65 mm, about 55 mm to about 70 mm, or about 55 mm to about 65 mm, from the 0 measurement. In some examples, a midpoint of the width of housing 110 may be about 40 mm, about 45 mm, about 50 mm, about 55 mm, about 60 mm, about 65 mm, or about 70 mm, from the 0 measurement.

A length of housing 110, represented by Measurement 6, may range from about 25 mm to about 75 mm. For example, a height of housing 110 may range from about 25 mm to about 70 mm, about 25 mm to about 65 mm, about 25 mm to about 60 mm, about 25 mm to about 55 mm, about 30 mm to about 75 mm, about 35 mm to about 75 mm, about 40 mm to about 75 mm, about 45 mm to about 75 mm, about 45 mm to about 70 mm, about 45 mm to about 65 mm, about 45 mm to about 60 mm, or about 45 mm to about 55 mm. In some examples, a length of housing 110 may be about 25 mm, about 30 mm, about 35 mm, about 40 mm, about 45 mm, about 50 mm, about 55 mm, about 60 mm, about 65 mm, about 70 mm, or about 75 mm.

A midpoint of the length of housing 110 is represented by Measurement 7. Measurement 7 is in reference to the 0 measurement, described above as the datum or start of the dimension. For example, a midpoint of the length of housing 110 may range from about 15 mm to about 35 mm, from the 0 measurement. For example, a midpoint of the length of housing 110 may range from about 15 mm to about 30 mm, about 15 mm to about 25 mm, about 20 mm to about 35 mm, about 20 mm to about 30 mm, or about 20 mm to about 25 mm, from the 0 measurement. In some examples, a midpoint of the length of housing 100 may be about 15 mm, about 20 mm, about 25 mm, about 30 mm, or about 35 mm, from the 0 measurement.

As discussed above, one of first plurality of walls 112a, 112b, may include slot 118 for receiving syringe needle 210. Referring to FIG. 5, wall 112a may include a first plurality of sidewalls 113a, 113b, and a second plurality of sidewalls 115a, 115b, wherein first plurality of sidewalls 113a, 113b correspond to Measurement 4 and second plurality of sidewalls 115a, 115b correspond to Measurement 2. Wall 112a may include slot 118, wherein slot 118 may extend from one of first plurality of sidewalls 113a, 113b to the second of first plurality of sidewalls 113a, 113b. As shown in FIG. 5, slot 118 may include a circular opening 119 for receiving a portion of syringe needle 210.

A width of slot 118 may range from about 2 mm to about 10 mm. For example, a width of slot 118 may range from about 2 mm to about 8 mm, about 2 mm to about 6 mm, about 3 mm to about 10 mm, about 3 mm to about 8 mm, about 3 mm to about 6 mm, about 4 mm to about 10 mm, about 4 mm to about 8 mm, or about 4 mm to about 6 mm. In some examples, a width of slot 118 may be about 2 mm, about 3 mm, about 4 mm, about 5 mm, about 6 mm, about 7 mm, about 8 mm, about 9 mm, or about 10 mm.

Slot 118 may be an opening, i.e., a gap, in wall 112a. Referring to FIG. 5, where slot 118 includes circular opening 119, a slot 118 may include a first ledge 118a and a second ledge 118b. Each of first ledge 118a and second ledge 118b may have a width represented by Measurement 5, the width ranging from about 2 mm to about 5 mm. For example, a width of each first ledge 118a and 118b may range from about 2 mm to about 4 mm, about 2 mm to about 3 mm, about 3 mm to about 4 mm, or about 4 mm to about 5 mm. In some examples, a width of each first ledge 118a and second ledge 118b may be about 2 mm, about 2.5 mm, about 3 mm, about 3.5 mm, about 4 mm, about 4.5 mm, or about 5 mm. A width of first ledge 118a may be equivalent to a width of second ledge 118b. In some examples, a width of first ledge 118a may differ from a width of second ledge 118b. As shown in FIG. 5, a radius of curvature of opening 119, represented by Measurement 3, may range from about 6 mm to about 14 mm. The radius of curvature of opening 119 may range from about 6 mm to about 13 mm, about 6 mm to about 12 mm, about 7 mm to about 14 mm, about 7 mm to about 13 mm, about 7 mm to about 12 mm, about 7 mm to about 11, about 8 mm to about 14 mm, about 8 mm to about 13 mm, about 8 mm to about 12 mm, about 8 mm to about 11 mm, about 9 mm to about 14 mm, about 9 mm to about 13 mm, about 9 mm to about 12 mm, or about 9 mm to about 11 mm. In some examples, the radius of curvature of opening 119 may be about 6 mm, about 7 mm, about 8 mm, about 9 mm, about 10 mm, about 11 mm, about 12 mm, about 13 mm, or about 14 mm.

An inner diameter of opening 119, represented by Measurement 1, may range from about 3.5 mm to about 9.5 mm. The inner diameter of opening 119 may range from about 3.5 mm to about 9 mm, about 3.5 mm to about 8.5 mm, about 3.5 mm to about 8 mm, about 3.5 mm to about 7.5 mm, about 3.5 mm to about 7 mm, about 4 mm to about 9.5 mm, about 4 mm to about 9 mm, about 4 mm to about 8.5 mm, about 4 mm to about 8 mm, about 4 mm to about 7.5 mm, about 4 mm to about 7 mm, about 4.5 mm to about 9.5 mm, about 4.5 mm to about 9 mm, about 4.5 mm to about 8.5 mm, about 4.5 mm to about 8 mm, about 4.5 mm to about 7.5 mm, about 4.5 mm to about 7 mm, about 5 mm to about 9.5 mm, about 5 mm to about 9 mm, about 5 mm to about 8.5 mm, about 5 mm to about 8 mm, about 5 mm to about 7.5 mm, about 5 mm to about 7 mm, about 5.5 mm to about 9.5 mm, about 5.5 mm to about 9 mm, about 5.5 mm to about 8.5 mm, about 5.5 mm to about 8 mm, about 5.5 mm to about 7.5 mm, about 5.5 mm to about 7 mm, around 6 mm to about 9.5 mm, about 6 mm to about 9 mm, about 6 mm to about 8.5 mm, about 6 mm to about 8 mm, about 6 mm to about 7.5 mm, or about 6 mm to about 7 mm.

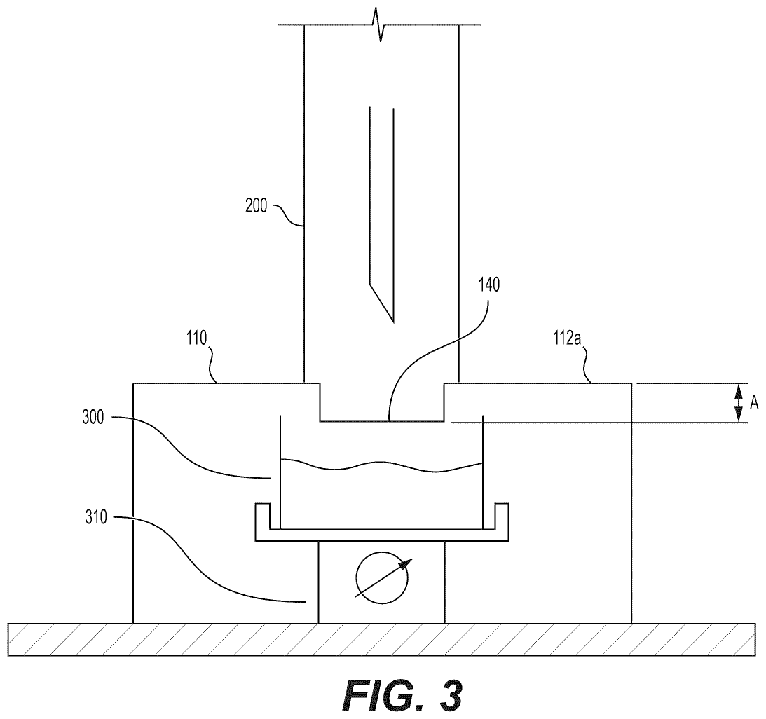

In some embodiments, chamber 130 may extend from one of the third plurality of walls 116a to a second of the third plurality of walls 116b (FIGS. 1, 2A, and 4B). In some embodiments, chamber 130 may be configured to hold a container 300 (shown in FIG. 3). Container 300 may be a depository for the drugs and/or samples expelled from the syringe during testing. In some embodiments, chamber 130 may have a continuous wall 132 (FIG. 4B). Container 300 may be placed atop a portion of continuous wall 132. Continuous wall 132 may be any appropriate size or shape to hold container 300. In some examples, continuous wall 132 may include an attachment means, e.g., an adhesive, clamp, or suitable geometric features, for receiving or securing container 300 in place.

The dimensions of chamber 130 are variable dependent on the type of container 300 to be held in chamber 130. Container 300 may have a desired volume and geometry, such that the dimensions of chamber 130 are adjustable to hold such container 300. In some examples, container 300 may be made from any appropriate material, e.g., plastic, and have any appropriate volume, e.g., ranging from about 10 mL to about 500 mL. A volume of container 300 may range from about 10 mL to about 450 mL, about 10 mL to about 400 mL, about 10 mL to about 350 mL, about 10 mL to about 300 mL, about 10 mL to about 250 mL, about 10 mL to about 200 mL, about 10 mL to about 150 mL, about 50 mL to about 500 mL, about 50 mL to about 450 mL, about 50 mL to about 400 mL, about 50 mL to about 350 mL, about 50 mL to about 300 mL, about 50 mL to about 250 mL, about 50 mL to about 200 mL, or about 50 mL to about 150 mL. In some examples, the volume of container 300 may be about 10 mL, 20 mL, about 30 mL, about 40 mL, about 50 mL, about 60 mL, about 70 mL, about 80 mL, about 90 mL, about 100 mL, about 150 mL, about 200 mL, about 250 mL, about 300 mL, about 350 mL, about 400 mL, about 450 mL, or about 500 mL.

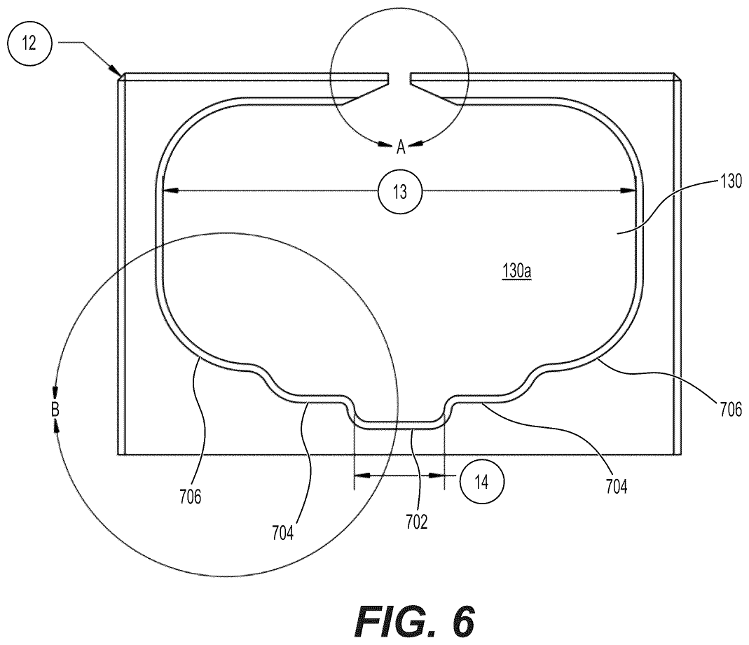

Referring to FIG. 6, chamber 130 may have an opening 130a. Opening 130a may have a maximum width, represented by Measurement 13, ranging from about 80 mm to about 120 mm. For example, opening 130a may have a maximum width ranging from about 80 mm to about 115 mm, about 80 mm to about 110 mm, about 85 mm to about 120 mm, about 85 mm to about 115 mm, about 85 mm to about 110 mm, about 85 mm to about 105 mm, about 90 mm to about 120 mm, about 90 mm to about 115 mm, about 90 mm to about 110 mm, about 95 mm to about 120 mm, about 95 mm to about 115 mm, about 95 mm to about 110 mm, about 100 mm to about 120 mm, about 100 mm to about 115 mm, or about 100 mm to about 110 mm. FIG. 6 also depicts a chamfer on the outer edge of the fixture. The chamfer is represented by Measurement 12. A chamfer is a transitional edge between two faces of a fixture. As shown in FIG. 6, the angle of the chamfer ranges from about 35° to about 55°, for example, from about 35° to about 50°, about 35° to about 45°, about 40° to about 55°, about 40° to about 50°, about 40° to about 45°, about 45° to about 55°, about 45° to about 50°, or about 50° to about 55°. For example, the angle the chamfer is about 35°, about 40°, about 45°, about 50°, or about 55°.

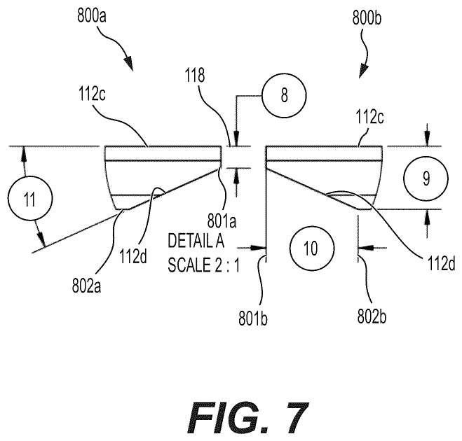

FIG. 7 depicts a detailed view of portion A from FIG. 6, including wall 112a and slot 118. The width of slot 118 is described in detail above. Referring to FIG. 7, wall 112a may include a first portion 800a and a second portion 800b, wherein first portion 800a and second portion 800b are separated by slot 118. First portion 800a and second portion 800b may each include a narrow portion 801a, 801b. Narrow portion 801a, 801b have a thickness, represented by Measurement 8, ranging from about 1 mm to about 4 mm. For example, each narrow portion 801a, 801b may have a thickness ranging from about 1 mm to about 3.6 mm, about 1 mm to about 3.2 mm, about 1 mm to about 2.8 mm, about 1.4 mm to about 4 mm, about 1.4 mm to about 3.6 mm, about 1.4 mm to about 3.2 mm, about 1.4 mm to about 2.8 mm, about 1.8 mm to about 4 mm, about 1.8 mm to about 3.6 mm, about 1.8 mm to about 3.2 mm, about 1.8 mm to about 2.8 mm, about 2.2 mm to about 4 mm, about 2.2 mm to about 3.6 mm, about 2.2 mm to about 3.2 mm, or about 2.2 mm to about 2.8 mm. In some examples, a thickness of narrow portion 801a, 801b may be about 1 mm, about 1.2 mm, about 1.4 mm, about 1.6 mm, about 1.8 mm, about 2 mm, about 2.2 mm, about 2.4 mm, about 2.6 mm, about 2.8 mm, about 3 mm, about 3.2 mm, about 3.4 mm, about 3.6 mm, about 3.8 mm, or about 4 mm.

First portion 800a and second portion 800b may each include a wide portion 802a, 802b. Wide portion 802a, 802b have a thickness, represented by Measurement 9, ranging from about 4 mm to about 10 mm. For example, each wide portion 802a, 802b may have a thickness ranging from about 4 mm to about 9 mm, about 4 mm to about 8 mm, about 5 mm to about 10 mm, about 5 mm to about 9 mm, about 5 mm to about 8 mm, about 6 mm to about 10 mm, about 6 mm to about 9 mm, or about 6 mm to about 8 mm. In some examples, each wide portion 802a, 802b may have a thickness of about 4 mm, about 5 mm, about 6 mm, about 7 mm, about 8 mm, about 9 mm, or about 10 mm.

A portion of wall 112a may slope, angle, or incline, towards slot 118. For example, wall 112a may include a first surface 112c and a second surface 112d. Referring to FIG. 7, a portion of second surface 112d may slope, angle, or incline, from narrow portion 801a, 801b to wide portion 802a, 802b. The length of second surface 112d between wide portion 802a, 802b and narrow portion 801a, 801b, represented by Measurement 10, may range from about 8 mm to about 12 mm. For example, the length of second surface 112d represented by Measurement 10 may range from about 8 mm to about 11.5 mm, about 8 mm to about 11 mm, about 8 mm to about 10.5 mm, about 8.5 mm to about 12 mm, about 8.5 mm to about 11.5 mm, about 8.5 mm to about 11 mm, about 8.5 mm to about 10.5 mm, about 9 mm to about 12 mm, about 9 mm to about 11.5 mm, about 9 mm to about 11 mm, about 9 mm to about 10.5 mm, about 9.5 mm to about 12 mm, about 9.5 mm to about 11.5 mm, about 9.5 mm to about 11 mm, about 9.5 mm to about 10.5 mm, about 10 mm to about 11 mm, or about 10 mm to about 10.55 mm. As shown in FIG. 7, a portion of second surface 112d, represented by Measurement 10, may angle away from first surface 112c. The angle of second surface 112d from first surface 112c is represented by Measurement 11 and may range from about 20° to about 45°. For example, the angle of second surface 112d from first surface 112c may range from about 20° to about 40°, about 20° to about 35°, about 20° to about 30°, or about 20° to about 25°.

As shown in FIGS. 4B and 6, continuous wall 132 of chamber 130 may include a plurality of ridges configured such that container 300 may stop atop one or more of the plurality of ridges. Referring to FIG. 6, continuous wall 132 may include a first ridge 702. First ridge 702 may have a width ranging from about 10 mm to about 30 mm. For example, a width of first ridge 702 may range from about 10 mm to about 28 mm, about 10 mm, to about 26 mm, about 10 mm to about 24 mm, about 10 mm to about 22 mm, about 12 mm to about 30 mm, about 12 mm to about 28 mm, about 12 mm to about 26 mm, about 12 mm to about 24 mm, about 12 mm to about 22 mm, about 14 mm to about 30 mm, about 14 mm to about 28 mm, about 14 mm to about 26 mm, about 14 mm to about 24 mm, about 14 mm to about 22 mm, about 16 mm to about 30 mm, about 16 mm to about 28 mm, about 16 mm to about 26 mm, about 16 mm to about 24 mm, about 16 mm to about 22 mm, about 18 mm to about 30 mm, about 18 mm to about 28 mm, about 18 mm to about 26 mm, about 18 mm to about 24 mm, or about 18 mm to about 22 mm.

As shown in FIG. 6, continuous wall 132 may include a second ridge 704 and a third ridge 706. Second ridge 704 may include a first portion and a second portion, wherein there is a gap between the portions. Third ridge 706 may also include a first portion and a second portion, wherein there is a gap between the portions. As shown in FIG. 4B, container 300 may sit stop at least one of the plurality of ridges. For example, container 300 may be configured such that it sits atop second ridge 704 and extends from the first portion of the second ridge 704 to the second portion of the second ridge 704. In another example, container 300 may be configured such that it sits atop third ridge 706 and extends from the first portion of third ridge 706 to the second portion of third ridge 706.

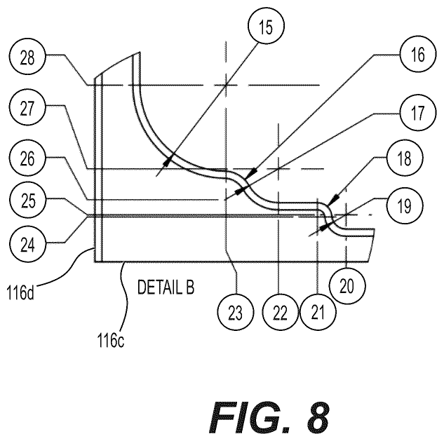

The measurements detailed in FIG. 8 are shown in reference to one side of chamber 130. The detailed description of the chamber 130, plurality of ridges, and measurements thereof, may be applied to the entire chamber 130 and the plurality of ridges in their entireties.

Continuous wall 132 may have a plurality of curvatures with corresponding radiuses of curvatures. A first radius of curvature, Measurement 15, may range from about 17 mm to about 21 mm. A first radius of curvature, Measurement 15, may range from about 17 mm to about 20 mm, about 18 mm to about 21 mm, about 18 mm to about 20 mm, or about 19 mm to about 21 mm. In some examples, a first radius of curvature, Measurement 15, may be about 17 mm, about 18 mm, about 19 mm, about 20 mm, or about 21 mm. In some examples, a first radius of curvature, Measurement 15, may be about 19.05 mm. A second radius of curvature, Measurement 16, may range from about 4 mm to about 8 mm. A second radius of curvature, Measurement 16, may range from about 4 mm to about 7 mm, about 5 mm to about 8 mm, about 5 mm to about 7 mm, about 6 mm to about 8 mm, or about 6 mm to about 7 mm. In some examples, a second radius of curvature, Measurement 16, may be about 4 mm, about 5 mm, about 6 mm, about 7 mm, or about 8 mm. In some examples, a second radius of curvature, Measurement 16, may be about 6.35 mm. A third radius of curvature, Measurement 17, may range from about 5 mm to about 9 mm. A third radius of curvature, Measurement 17, may range from about 5 mm to about 8 mm, about 6 mm to about 9 mm, about 6 mm to about 8 mm, or about 6 mm to about 7 mm. In some examples, a third radius of curvature, Measurement 17, may be about 5 mm, about 6 mm, about 7 mm, about 8 mm, or about 9 mm. In some examples, a third radius of curvature, Measurement 18, may be about 7.5 mm. A fourth radius of curvature, Measurement 18, may range from about 1 mm to about 5 mm. A fourth radius of curvature, Measurement 18, may range from about 1 mm to about 4 mm, about 2 mm to about 5 mm, about 2 mm to about 4 mm, about 3 mm to about 5 mm, or about 3 mm to about 4 mm. In some examples, a fourth radius of curvature, Measurement 18, may be about 1 mm, about 2 mm, about 3 mm, about 4 mm, or about 5 mm. In some examples, a fourth radius of curvature, Measurement 18, may be about 3.18 mm. A fifth radius of curvature, Measurement 19, may range from about 1 mm to about 5 mm. A fifth radius of curvature, Measurement 19, may range from about 1 mm to about 4 mm, about 2 mm to about 5 mm, about 2 mm to about 4 mm, about 3 mm to about 5 mm, or about 3 mm to about 4 mm. In some examples, a fifth radius of curvature, Measurement 19, may be about 1 mm, about 2 mm, about 3 mm, about 4 mm, or about 5 mm. In some examples, a fifth radius of curvature, Measurement 19, may be about 3.18 mm.

Referring to FIGS. 1, 2, and 8, second plurality of sidewalls 116a, 116b may each include a first plurality of sides 116c and a second plurality of sides 116d, wherein the first plurality of sides 116c may be longer than the second plurality of sides 116d. Referring to FIG. 8, Measurements 20, 21, 22, and 23, are in reference to side 116d and specify a distance that the designated portion of continuous wall 132 may be away from side 116d.

A length of Measurement 20 may range from about 46 mm to about 66 mm. For example, a length of Measurement 20 may range from about 46 mm to about 64 mm, about 46 mm to about 62 mm, about 46 mm to about 60 mm, about 46 mm to about 58 mm, about 48 mm to about 66 mm, about 48 mm to about 64 mm, about 48 mm to about 62 mm, about 48 mm to about 60 mm, about 48 mm to about 58 mm, about 50 mm to about 66 mm, about 50 mm to about 64 mm, about 50 mm to about 62 mm, about 50 mm to about 60 mm, about 50 mm to about 58 mm, about 52 mm to about 66 mm, about 52 mm to about 64 mm, about 52 mm to about 62 mm, about 52 mm to about 60 mm, about 52 mm to about 58 mm, about 54 mm to about 66 mm, about 54 mm to about 64 mm, about 54 mm to about 62 mm, about 54 mm to about 60 mm, about 54 mm to about 58 mm. In some examples, a length of Measurement 20 may be about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, or about 66 mm.

A length of Measurement 21 may range from about 39 mm to about 59 mm. For example, a length of Measurement 21 may range from about 39 mm to about 57 mm, about 39 mm to about 55 mm, about 39 mm to about 53 mm, about 39 mm to about 51 mm, about 41 mm to about 59 mm, about 41 mm to about 57 mm, about 41 mm to about 55 mm, about 41 mm to about 53 mm, about 41 mm to about 51 mm, about 43 mm to about 59 mm, about 43 mm to about 57 mm, about 43 mm to about 55 mm, about 43 mm to about 53 mm, about 43 mm to about 51 mm, about 45 mm to about 59 mm, about 45 mm to about 57 mm, about 45 mm to about 55 mm, about 45 mm to about 53 mm, about 45 mm to about 51 mm, about 47 mm to about 59 mm, about 47 mm to about 57 mm, about 47 mm to about 55 mm, about 47 mm to about 53 mm, or about 47 mm to about 51 mm. In some examples, a length of Measurement 21 may be about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, or about 59 mm.

A length of Measurement 22 may range from about 31 mm to about 51 mm. For example, a length of Measurement 22 may range from about 31 mm to about 49 mm, about 31 mm to about 47 mm, about 31 mm to about 45 mm, about 31 mm to about 43 mm, about 33 mm to about 51 mm, about 33 mm to about 49 mm, about 33 mm to about 47 mm, about 33 mm to about 45 mm, about 33 mm to about 43 mm, about 35 mm to about 51 mm, about 35 mm to about 49 mm, about 35 mm to about 47 mm, about 35 mm to about 45 mm, about 35 mm to about 43 mm, about 37 mm to about 51 mm, about 37 mm to about 49 mm, about 37 mm to about 47 mm, about 37 mm to about 45 mm, about 37 mm to about 43 mm, about 39 mm to about 51 mm, about 39 mm to about 49 mm, about 39 mm to about 47 mm, about 39 mm to about 45 mm, or about 39 mm to about 43 mm. In some examples, a length of Measurement 22 may be about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, or about 51 mm.

A length of Measurement 23 may range from about 19 mm to about 39 mm. For example, a length of Measurement 23 may range from about 19 mm to about 37 mm, about 19 mm to about 35 mm, about 19 mm to about 33 mm, about 19 mm to about 31 mm, about 21 mm to about 39 mm, about 21 mm to about 37 mm, about 21 mm to about 35 mm, about 21 mm to about 33 mm, about 21 mm to about 31 mm, about 23 mm to about 39 mm, about 23 mm to about 37 mm, about 23 mm to about 35 mm, about 23 mm to about 33 mm, about 23 mm to about 31 mm, about 25 mm to about 39 mm, about 25 mm to about 37 mm, about 25 mm to about 35 mm, about 25 mm to about 33 mm, about 25 mm to about 31 mm, about 27 mm to about 39 mm, about 27 mm to about 37 mm, about 27 mm to about 35 mm, about 27 mm to about 33 mm, or about 27 mm to about 31 mm. In some examples, a length of Measurement 23 may be about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, or about 39 mm.

Referring to FIG. 8, Measurements 24, 25, 26, 27, and 28, are in reference to side 116c and specify a distance that the designated portion of continuous wall 132 may be away from side 116c.

A height of Measurement 24 may range from about 8.95 mm to about 10.95 mm. For example, a height of Measurement 24 may range from about 8.95 mm to about 10.85 mm, about 8.95 mm to about 10.75 mm, about 8.95 mm to about 10.65 mm, about 8.95 mm to about 10.55 mm, about 8.95 mm to about 10.45 mm, about 8.95 mm to about 10.35 mm, about 8.95 mm to about 10.25 mm, about 8.95 mm to about 10.15 mm, about 8.95 mm to about 10.05 mm, about 9.05 mm to about 10.95 mm, about 9.05 mm to about 10.85 mm, about 9.05 mm to about 10.75 mm, about 9.05 mm to about 10.65 mm, about 9.05 mm to about 10.55 mm, about 9.05 mm to about 10.45 mm, about 9.05 mm to about 10.35 mm, about 9.05 mm to about 10.25 mm, about 9.05 mm to about 10.15 mm, about 9.05 mm to about 10.05 mm, about 9.15 mm to about 10.95 mm, about 9.15 mm to about 10.85 mm, about 9.15 mm to about 10.75 mm, about 9.15 mm to about 10.65 mm, about 9.15 mm to about 10.55 mm, about 9.15 mm to about 10.45 mm, about 9.15 mm to about 10.35 mm, about 9.15 mm to about 10.25 mm, about 9.15 mm to about 10.15 mm, about 9.15 mm to about 10.05 mm, about 9.25 mm to about 10.85 mm, about 9.25 mm to about 10.75 mm, about 9.25 mm to about 10.65 mm, about 9.25 mm to about 10.55 mm, about 9.25 mm to about 10.45 mm, about 9.25 mm to about 10.35 mm, about 9.25 mm to about 10.25 mm, about 9.25 mm to about 10.15 mm, about 9.25 mm to about 10.05 mm, about 9.35 mm to about 10.95 mm, about 9.35 mm to about 10.85 mm, about 9.35 mm to about 10.75 mm, about 9.35 mm to about 10.65 mm, about 9.35 mm to about 10.55 mm, about 9.35 mm to about 10.45 mm, about 9.35 mm to about 10.35 mm, about 9.35 mm to about 10.25 mm, about 9.35 mm to about 10.15 mm, about 9.35 mm to about 10.05 mm, about 9.45 mm to about 10.95 mm, about 9.45 mm to about 10.85 mm, about 9.45 mm to about 10.75 mm, about 9.45 mm to about 10.65 mm, about 9.45 mm to about 10.55 mm, about 9.45 mm to about 10.45 mm, about 9.45 mm to about 10.35 mm, about 9.45 mm to about 10.25 mm, about 9.45 mm to about 10.15 mm, about 9.45 mm to about 10.05 mm, about 9.55 mm to about 10.95 mm, about 9.55 mm to about 10.85 mm, about 9.55 mm to about 10.75 mm, about 9.55 mm to about 10.65 mm, about 9.55 mm to about 10.55 mm, about 9.55 mm to about 10.45 mm, about 9.55 mm to about 10.35 mm, about 9.55 mm to about 10.25 mm, about 9.55 mm to about 10.15 mm, about 9.55 mm to about 10.05 mm, about 9.65 mm to about 10.95, about 9.65 mm to about 10.85 mm, about 9.65 mm to about 10.75 mm, about 9.65 mm to about 10.65 mm, about 9.65 mm to about 10.55 mm, about 9.65 mm to about 10.45 mm, about 9.65 mm to about 10.35 mm, about 9.65 mm to about 10.25 mm, about 9.65 mm to about 10.15 mm, about 9.65 mm to about 10.05 mm, about 9.75 mm to about 10.95 mm, about 9.75 mm to about 10.85 mm, about 9.75 mm to about 10.75 mm, about 9.75 mm to about 10.65 mm, about 9.75 mm to about 10.55 mm, about 9.75 mm to about 10.45 mm, about 9.75 mm to about 10.35 mm, about 9.75 mm to about 10.25 mm, about 9.75 mm to about 10.15 mm, about 9.75 mm to about 10.05 mm, about 9.85 mm to about 10.95 mm, about 9.85 mm to about 10.85 mm, about 9.85 mm to about 10.75 mm, about 9.85 mm to about 10.65 mm, about 9.85 mm to about 10.55 mm, about 9.85 mm to about 10.45 mm, about 9.85 mm to about 10.35 mm, about 9.85 mm to about 10.25 mm, about 9.85 mm to about 10.15 mm, or about 9.85 mm to about 10.05 mm. In some examples, a height of Measurement 24 may be about 8.85 mm, about 8.95 mm, about 9.05 mm, about 9.15 mm, about 9.25 mm, about 9.35 mm, about 9.45 mm, about 9.55 mm, about 9.65 mm, about 9.75 mm, about 9.85 mm, about 9.95 mm, about 10.05 mm, about 10.15 mm, about 10.25 mm, about 10.35 mm, about 10.45 mm, about 10.55 mm, about 10.65 mm, about 10.75 mm, about 10.85 mm, or about 10.95 mm.

A height of Measurement 25 may range from about 6 mm to about 14 mm. For example, a height of Measurement 25 may range from about 6 mm to about 13 mm, about 6 mm to about 12 mm, about 6 mm to about 11 mm, about 7 mm to about 14 mm, about 7 mm to about 13 mm, about 7 mm to about 12 mm, about 7 mm to about 11 mm, about 8 mm to about 14 mm, about 8 mm to about 13 mm, about 8 mm to about 12 mm, about 8 mm to about 11 mm, about 9 mm to about 14 mm, about 9 mm to about 13 mm, about 9 mm to about 12 mm, or about 9 mm to about 11 mm. In some examples, a height of Measurement 25 may be about 6 mm, about 7 mm, about 8 mm, about 9 mm, about 10 mm, about 11 mm, about 12 mm, about 13 mm, or about 14 mm.

A height of Measurement 26 may range from about 8 mm to about 20 mm. For example, a height of Measurement 26 may range from about 8 mm to about 19 mm, about 8 mm to about 18 mm, about 8 mm to about 17 mm, about 8 mm to about 16 mm, about 8 mm to about 15 mm, about 9 mm to about 20 mm, about 9 mm to about 19 mm, about 9 mm to about 18 mm, about 9 mm to about 17 mm, about 9 mm to about 16 mm, about 9 mm to about 15 mm, about 10 mm to about 20 mm, about 10 mm to about 19 mm, about 10 mm to about 18 mm, about 10 mm to about 17 mm, about 10 mm to about 16 mm, about 10 mm to about 15 mm, about 11 mm to about 20 mm, about 11 mm to about 19 mm, about 11 mm to about 18 mm, about 11 mm to about 17 mm, about 11 mm to about 16 mm, about 11 mm to about 15 mm, about 12 mm to about 20 mm, about 12 mm to about 19 mm, about 12 mm to about 18 mm, about 12 mm to about 17 mm, about 12 mm to about 16 mm, about 12 mm to about 15 mm, about 13 mm to about 20 mm, about 13 mm to about 19 mm, about 13 mm to about 18 mm, about 13 mm to about 17 mm, about 13 mm to about 16 mm, or about 13 mm to about 15 mm. In some examples, a height of Measurement 26 may be about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm.

A height of Measurement 27 may range from about 11 mm to about 31 mm. For example, a height of Measurement 27 may range from about 11 mm to 29 mm, about 11 mm to about 27 mm, about 11 mm to about 25 mm, about 11 mm to about 23 mm, about 13 mm to about 31 mm, about 13 mm to about 29 mm, about 13 mm to about 27 mm, about 13 mm to about 25 mm, about 13 mm to about 23 mm, about 15 mm to about 31 mm, about 15 mm to about 29 mm, about 15 mm to about 27 mm, about 15 mm to about 25 mm, about 15 mm to about 23 mm, about 17 mm to about 31 mm, about 17 mm to about 29 mm, about 17 mm to about 27 mm, about 17 mm to about 25 mm, about 17 mm to about 23 mm, about 19 mm to about 31 mm, about 19 mm to about 29 mm, about 19 mm to about 27 mm, about 19 mm to about 25 mm, or about 19 mm to about 23 mm. In some examples, a height of Measurement 27 may be about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, or about 31 mm.

A height of Measurement 28 may range from about 29 mm to about 49 mm. For example, a height of Measurement 28 may range from about 29 mm to about 47 mm, about 29 mm to about 45 mm, about 29 mm to about 43 mm, about 29 mm to about 41 mm, about 31 mm to about 49 mm, about 33 mm to about 47 mm, about 33 mm to about 45 mm, about 33 mm to about 43 mm, about 33 mm to about 41 mm, about 35 mm to about 49 mm, about 35 mm to about 47 mm, about 35 mm to about 45 mm, about 35 mm to about 43 mm, about 35 mm to about 41 mm, about 37 mm to about 49 mm, about 37 mm to about 47 mm, about 35 mm to about 45 mm, about 35 mm to about 43 mm, or about 35 mm to about 41 mm. In some examples, a height of Measurement 28 may be about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, or about 49 mm.

Fixture 100 may also include a membrane holder 120. Membrane holder 120 may be any suitable holder, feeder, or configuration such that a sample membrane 140 may be properly secured and held with sufficient tension to allow for proper puncture testing. Membrane holder 120 may also be configured such that membrane 140 automatically feeds through housing 110. Membrane holder 120 may be any device, e.g., a disc, wheel, cassette, cartridge, or any appropriate device that may rotate membrane 140 and/or feed membrane 140 through the device such that a new, unused portion of membrane 140 is positioning for puncturing in each test run. Referring to FIGS. 1, 2A, and 2B, membrane holder 120 may have a circular shape, e.g., a disc shape, comprising a first portion 122 and a second portion 124. Membrane 140 may be clamped between first portion 122 and second portion 124 (FIGS. 2A and 2B). For illustrative purposes, FIG. 2A shows membrane holder 120 as it would be viewed through one of the first plurality of walls 112a. In some embodiments, membrane holder 120 may include a plurality of holes 126a, 126b of various sizes. Plurality of holes 126a, 126b may be configured to receive fastening or attachments means, e.g., screws. Plurality of holes 126a, 126b may serve various purposes. For example, plurality of holes 126a, 126b may be used to clamp membrane 140 between first portion 122 and second portion 124, secure membrane holder 120 to housing 110, and/or allow for rotation of membrane holder 120.

Plurality of holes 126a, 126b may any size, shape, or configuration, to properly receive fastening of attachment means. Each of plurality of holes 126a may have a diameter ranging from about 2 mm to about 10 mm. For example, each of plurality of holes 126a may have a diameter ranging from about 2 mm to about 9 mm, about 2 mm to about 8 mm, about 2 mm to about 7 mm, about 3 mm to about 10 mm, about 3 mm to about 9 mm, about 3 mm to about 8 mm, about 3 mm to about 7 mm, about 4 mm to about 10 mm, about 4 mm to about 9 mm, about 4 mm to about 8 mm, about 4 mm to about 7 mm, about 5 mm to about 10 mm, about 5 mm to about 9 mm, about 5 mm to about 8 mm, or about 5 mm to about 7 mm. For example, each of plurality of holes 126a may have a diameter of about 2 mm, about 3 mm, about 4 mm, about 5 mm, about 6 mm, about 7 mm, about 8 mm, about 9 mm, or about 10 mm.

Each of plurality of holes 126b may have a diameter ranging from about 1 mm to about 8 mm. For example, each of plurality of holes 126b may have a diameter ranging from about 1 mm to about 7 mm, about 1 mm to about 6 mm, about 1 mm to about 5 mm, about 2 mm to about 8 mm, about 2 mm to about 7 mm, about 2 mm to about 6 mm, about 2 mm to about 5 mm, about 3 mm to about 8 mm, about 3 mm to about 7 mm, about 3 mm to about 6 mm, or about 3 mm to about 5 mm. For example, each of plurality of holes 126b may have a diameter of about 1 mm, about 2 mm, about 3 mm, about 4 mm, about 5 mm, about 6 mm, about 7 mm, or about 8 mm. In some embodiments, at least one of first portion 122 and second portion 124 may include a plurality of cutouts 128. Plurality of cutouts 128 may be configured to allow syringe needle 210 to penetrate through membrane 140. Plurality of cutouts 128 may be any appropriate size or shape to allow syringe needle 210 to penetrate membrane 140 without interfering with or inhibiting movement of syringe needle 210. Plurality of cutouts 128 may be in a radial shape (FIGS. 2A and 2B), a circular shape, or a rectangular shape. In some embodiments, both first portion 122 and second portion 124 may include a plurality of cutouts 128 corresponding to one another. In such examples, a first plurality of cutouts 129a may extend through first portion 122 and a second plurality of cutouts 129b may extend through second portion 124 (FIG. 2B). Referring to FIG. 2B, first plurality of cutouts 129a in first portion 122 and second plurality of cutouts 129b in second portion 124 may align such that syringe needle 210 may penetrate through membrane 140, allowing for syringe needle 210 to be viewed after it penetrates through membrane 140 and into chamber 130 (FIG. 1). To better depict first plurality of cutouts 129a and second plurality of cutouts 129b, membrane holder 120 in FIG. 2B is illustrated without membrane 140. Configuring membrane holder 120 such that syringe needle 210 may penetrate through membrane 140, may allow the user to obtain proper images and/or data during the puncture test.

As mentioned above, plurality of cutouts 128 may have a size, shape, and/or configuration to allow syringe needle 210 to penetrate membrane 140 without interfering with or inhibiting movement of syringe needle 210. As shown in FIG. 2B, exemplary plurality of cutouts 128 may have a radial shape. Each of the rays may have a length ranging from about 10 mm to about 50 mm. For example, each of the rays may have a length ranging from about 10 mm to about 45 mm, about 10 mm to about 40 mm, about 10 mm to about 35 mm, about 15 mm to about 50 mm, about 15 mm to about 45 mm, about 15 mm to about 40 mm, about 15 mm to about 35 mm, about 20 mm to about 50 mm, about 20 mm to about 45 mm, about 20 mm to about 40 mm, about 20 mm to about 35 mm, about 25 mm to about 50 mm, about 25 mm to about 45 mm, about 25 mm to about 40 mm, or about 25 mm to about 35 mm. In some examples, each of the rays may have a length of about 10 mm, about 15 mm, about 20 mm, about 25 mm, about 30 mm, about 35 mm, about 40 mm, about 45 mm, or about 50 mm. In some examples, each of the rays may have various lengths. In other examples, each of the rays may have identical lengths.

Each of the rays may have a width ranging from about 1 mm to about 10 mm. For example, each of the rays may have a width ranging from about 1 mm to about 9 mm, about 1 mm to about 8 mm, about 1 mm to about 7 mm, about 1 mm to about 6 mm, about 1 mm to about 5 mm, about 2 mm to about 10 mm, about 2 mm to about 9 mm, about 2 mm to about 8 mm, about 2 mm to about 7 mm, about 2 mm to about 6 mm, about 2 mm to about 5 mm, about 3 mm to about 10 mm, about 3 mm to about 9 mm, about 3 mm to about 8 mm, about 3 mm to about 7 mm, or about 3 mm to about 5 mm. In some examples, each of the rays may have a width of about 1 mm, about 2 mm, about 3 mm, about 4 mm, about 5 mm, about 6 mm, about 7 mm, about 8 mm, about 9 mm, or about 10 mm. Membrane holder 120 may also be configured to properly hold membrane 140 during testing. As discussed above, potential issues arise with membrane testing, e.g., puncture testing, as the membrane may not be securely and/or tightly held, such that the membrane may lose tension and fold into itself upon contact from the syringe needle. A deformed membrane during testing will produce inaccurate results. As such, it is important to have the membrane be taut and maintain its optimal tension during testing. In the disclosed embodiments, membrane 140 may be secured between first portion 122 and second portion 124 of membrane holder 120. Placing membrane 140 in membrane holder 120 may provide a properly taut and secured membrane 140 for puncture testing.

Tension may be measured by a handheld durometer tool, a Zeiss O-Inspect multi-sensor measuring machine, or any other appropriate methods or devices. The tension of membrane 140 in membrane holder 120 may range from about 2 N to about 100 N. For example, the tension of membrane 140 in membrane holder 120 may range from about 2 N to about 90 N, about 2 N to about 80 N, about 2 N to about 70 N, about 2 N to about 60 N, about 2 N to about 50 N, about 2 N to about 40 N, about 2 N to about 30 N, about 2 N to about 20 N.

To satisfy testing requirements and obtain an adequate number of results, numerous test runs may be performed. In other words, during puncture testing, numerous punctures may need to be made in the membrane. As discussed above, the membrane cannot be punctured in the same place repeatedly. Each puncture should be made in an unused portion of the membrane to ensure testing integrity. Current test systems are not configured to automatically move the membrane so that the syringe needle can puncture an unused portion of the membrane for each test run. In addition, each puncture should be made a sufficient distance away from prior punctures so as to not weaken or destroy the membrane. A weakened membrane may droop and/or break during testing, preventing a proper puncture and/or undesirable penetration levels/results. While a user may manually move the membrane for each test run, such that an unused portion is ready for puncture and/or the new injection site is sufficiently far from prior injection sites, this would negatively affect efficiency and user error may be introduced into the test. Moreover, manually moving the membrane and/or locating unused portions of the membrane may not be precise, thereby introducing inefficiencies and nonuniformity in the number of punctures per membrane.

In the disclosed embodiments, fixture 100 may be operatively coupled to the test system such that membrane holder 120 automatically moves after each test run, allowing syringe needle 210 to puncture an unused portion of membrane 140. Fixture 100 may utilize any appropriate means to automatically move membrane holder 120. For example, fixture 100 may include a motor or a solenoid for automatically moving membrane holder 120. In embodiments wherein membrane holder 120 is a wheel (FIGS. 2A and 2B), membrane holder 120 may rotate after each test run such that an unused portion of membrane 140 may be punctured by syringe needle 210.

As mentioned above, it is also important that each puncture is made a sufficient distance away from prior punctures so as to not unnecessarily weaken or damage the membrane. Plurality of cutouts 128 may be configured such that each of the cutouts are spaced apart at an appropriate distance. For example, referring to FIGS. 2A and 2B, at least one puncture may be made in each of the radial arms of plurality of cutouts 128. In some embodiments, membrane holder 120 may include markings and/or codes, e.g., grid lines, axes, color codes, etc. to designate the various positions of potential injection sites. The use of membrane holder 120, including first portion 122, second portion 124, and membrane 140, may produce up to about 30 different injection sites. For example, membrane holder 120 may include up to about 10 different injection sites, about 15 different injection sites, about 20 different injection sites, or about 25 different injection sites. Membrane holder 120 may include from about 5 to about 30 different injection sites, about 5 to about 25 different injection sites, about 5 to about 20 different injection sites, or about 5 to about 15 different injection sites. In other words, membrane 140 may be punctured a number of times ranging from about 5 times to about 30 times. For example, membrane 140 may be punctured a number of times ranging from about 5 times to about 25 times, about 5 times to about 20 times, or about 5 times to about 15 times. For example, membrane 140 may be punctured about 5 times, about 6 times, about 7 times, about 8 times, about 9 times, about 10 times, about 11 times, about 12 times, around 13 times, about 14 times, about 15 times, about 16 times, about 17 times, about 18 times, about 19 times, about 20 times, about 21 times, about 22 times, about 23 times, about 24 times, about 25 times, about 26 times, about 27 times, about 28 times, about 29 times, or about 30 times.

As mentioned above, updates to ISO 11608 now require testing of syringes to ensure dosing of a medicament or collection of a sample occurs at about 4 mm below the injection surface. In other words, once a syringe needle penetrates an injection site, the syringe needle should advance to about 4 mm below the surface of the injection site prior to dosing and/or collection of a sample. In some embodiments, the desired dosage/collection depth may range from about 4 mm to about 8 mm below the surface of the injection site. A depth ranging from about 4 mm to about 8 mm represents where subcutaneous tissue may be located in a patient's injection site. After the syringe needle reaches the desired depth, i.e., at least about 4 mm below the surface of the injection site, dosage or collection may occur. To comply with this new requirement, the puncture test analyzes the extension of the syringe needle and how deep the syringe needle extends before dosage/collection occurs.

Embodiments of the present disclosure may be designed to mimic the desired dosage/collection depth. Referring to FIGS. 1 and 2A, a portion of syringe 200 abuts a top surface of housing 110. As shown in FIG. 1, a portion of syringe 200 abuts one of first plurality of walls 112a. This surface of wall 112a represents the injection site. Syringe needle 210 may puncture membrane 140 and be viewed after it has passed through membrane holder 120 and membrane 140 (FIG. 1). A height of membrane holder 120 may be chosen such that membrane 140 is secured at about 4 mm below the surface of the injection site. Membrane holder 120 may have a height ranging from about 2 mm to about 10 mm. For example, membrane holder 120 may have a height ranging from about 2 mm to about 9 mm, about 2 mm to about 8 mm, about 2 mm to about 7 mm, about 2 mm to about 6 mm, about 2 mm to about 5 mm, about 3 mm to about 10 mm, about 3 mm to about 9 mm, about 3 mm to about 8 mm, about 3 mm to about 7 mm, about 3 mm to about 6 mm, or about 3 mm to about 5 mm. In some examples, membrane holder 120 may have a height of about 2 mm, about 3 mm, about 4 mm, about 5 mm, about 6 mm, about 7 mm, about 8 mm, about 9 mm, or about 10 mm.

In some embodiments, first portion 122 may have a height ranging from about 1 mm to about 9 mm. For example, first portion 122 may have a height ranging from about 1 mm to about 8 mm, about 1 mm to about 7 mm, about 1 mm to about 6 mm, about 1 mm to about 5 mm, about 1 mm to about 4 mm, about 1 mm to about 3 mm, about 2 mm to about 9 mm, about 2 mm to about 8 mm, about 2 mm to about 7 mm, about 2 mm to about 6 mm, about 2 mm to about 5 mm, about 2 mm to about 4 mm, about 3 mm to about 9 mm, about 3 mm to about 8 mm, about 3 mm to about 7 mm, about 3 mm to about 6 mm, about 3 mm to about 5 mm, about 4 mm to about 9 mm, about 4 mm to about 8 mm, about 4 mm to about 7 mm, about 4 mm to about 6 mm, about 5 mm to about 9 mm, about 5 mm to about 8 mm, about 5 mm to about 7 mm, about 6 mm to about 9 mm, about 6 mm to about 8 mm, or about 7 mm to about 9 mm. In some examples, first portion 122 may have a height of about 1 mm, about 2 mm, about 3 mm, about 4 mm, about 5 mm, about 6 mm, about 7 mm, about 8 mm, or about 9 mm.

In some embodiments, second portion 124 may have a height ranging from about 1 mm to about 9 mm. For example, second portion 124 may have a height ranging from about 1 mm to about 8 mm, about 1 mm to about 7 mm, about 1 mm to about 6 mm, about 1 mm to about 5 mm, about 1 mm to about 4 mm, about 1 mm to about 3 mm, about 2 mm to about 9 mm, about 2 mm to about 8 mm, about 2 mm to about 7 mm, about 2 mm to about 6 mm, about 2 mm to about 5 mm, about 2 mm to about 4 mm, about 3 mm to about 9 mm, about 3 mm to about 8 mm, about 3 mm to about 7 mm, about 3 mm to about 6 mm, about 3 mm to about 5 mm, about 4 mm to about 9 mm, about 4 mm to about 8 mm, about 4 mm to about 7 mm, about 4 mm to about 6 mm, about 5 mm to about 9 mm, about 5 mm to about 8 mm, about 5 mm to about 7 mm, about 6 mm to about 9 mm, about 6 mm to about 8 mm, or about 7 mm to about 9 mm. In some examples, second portion 124 may have a height of about 1 mm, about 2 mm, about 3 mm, about 4 mm, about 5 mm, about 6 mm, about 7 mm, about 8 mm, or about 9 mm.

A height of first portion 122 and a height of second portion 124 may be chosen such that membrane 140 may be located at a desired depth for the puncture test. In some embodiments, a height of first portion 122 and a height of second portion 124 may be chosen such that membrane 140 may be about 4 mm before the surface of the injection site.

Membrane 140 may be any material that mimics human tissue, e.g., subcutaneous tissue. In some embodiments, membrane 140 may comprise plastic, polyurethane, or both. Membrane 140 may have any appropriate size and shape to fit into membrane holder 120. In embodiments where membrane holder 120 is a disc or wheel, membrane 140 may have a circular shape to fit into the disc or wheel (FIGS. 2A and 2B). In other embodiments where membrane holder 120 may be a cartridge or a cassette, membrane 140 may be provided as a continuous strip or roll that may be fed through the cartridge or cassette.

Membrane 140 may have any appropriate thickness such that membrane 140 may be used with membrane holder 120. Membrane 140 may have a thickness ranging from about 0.1 mm to about 1.0 mm. In some examples, membrane 140 may have a thickness ranging from about 0.1 mm to about 0.9 mm, about 0.1 mm to about 0.8 mm, about 0.1 mm to about 0.7 mm, about 0.1 mm to about 0.6 mm, about 0.1 mm to about 0.5 mm, about 0.2 mm to about 0.9 mm, about 0.2 mm to about 0.8 mm, about 0.2 mm to about 0.7 mm, about 0.2 mm to about 0.6 mm, about 0.2 mm to about 0.5 mm, about 0.3 mm to about 0.9 mm, about 0.3 mm to about 0.8 mm, about 0.3 mm to about 0.7 mm, about 0.3 mm to about 0.6 mm, or about 0.3 mm to about 0.5 mm. In some examples, membrane 140 may have a thickness of about 0.1 mm, about 0.2 mm, about 0.3 mm, about 0.4 mm, about 0.5 mm, about 0.6 mm, about 0.7 mm, about 0.8 mm, about 0.9 mm, or about 1.0 mm.

FIG. 3 further shows the use of fixture 100 in a test system to analyze syringe 200. The embodiment of FIG. 3 may include a membrane holder 120 (not shown) such that membrane 140 is located at a distance below the top surface of one of the first plurality of walls 112a, represented by dimension A. Dimension A may correspond to a minimum intended injection depth, ranging from about 2 mm to about 6 mm or about 3 mm to about 5 mm. In some examples, dimension A may be about 2 mm, 3 mm, about 4 mm, about 5 mm, or about 6 mm. FIG. 3 also depicts container 300 sitting atop a scale 310. Scale 310 may be used to weigh the medicament/sample expelled from the device, i.e., syringe needle 210, during testing.

In some embodiments, fixture 100 may be secured to a mechanical test system, e.g., an Instron® test system or a ZwickRoell test system. For attachment to a test system, at least one of the first plurality of walls 112a, 112b may include an attachment means. Referring to FIG. 4B, one of the first plurality of walls 112b may include a fastener 542, e.g., a screw, for further attachment to a portion of the test system. Referring to FIG. 4B, housing 110 may be attached to a base 540 of the test system.

In some embodiments, a component of test system 500 and/or fixture 100 may be controllable by a controller, e.g., a digital controller, such as a programmable logic controller. In some embodiments, the controller may be pre-programmed to execute one or more puncture tests using test system 500. In some embodiments, test system 500 may be controllable by a controller having one or more human machine interface (“HMI”) elements, which may be configured to allow a user to input or alter desired parameters for a puncture test, which may be executable by a controller on or operably coupled to a test system. Thus, in some embodiments, HMI elements may be used to program a customized puncture test for execution by test system 500. For example, in some embodiments, test system 500 may be controllable by a controller connected to, e.g., a computer, tablet, or handheld device having a display. In some embodiments, fixture 100 may be connected to test system 500, e.g., an Instron® test system or a ZwickRoell test system, such that membrane holder 120 may be moved and/or rotated after each test run allowing for a new, unused portion of membrane 140 to be punctured.

The following example is provided for illustrative purposes only and is not intended to limit any single aspect or embodiment of the fixture, nor is it limited to any combinations and/or permutations of such aspects and/or embodiments. This example illustrates how the fixture of the present disclosure can be used with a dose precision system syringe that is described in WO 2019/118588 or WO 2020/247686 and/or with a mechanical test system, e.g., an Instron® test system or a ZwickRoell test system. A person skilled in the art would appreciate that the fixture can be adapted to fit other types of syringes and test systems and that other measurements are possible given various applications and/or medicaments disposed within the syringe.

An exemplary use of fixture embodiments as disclosed is provided herein. A fixture is inserted into and attached to an Instron® test system 5943, e.g., as shown in FIGS. 4A and 4B. Housing 110 and membrane holder 120 may be fixed on the Instron test system. An autoinjector may then be placed in a clamp 520 that secures the autoinjector within syringe housing 510 of test system 500. Container 300 may be tared on a scale or balance and then placed into housing 110. Appropriate steps may be taken to ensure membrane 140 is properly tensioned within membrane holder 120. The Instron test system may be initiated, causing a load string of the Instron system to move and activate the syringe. After activation, the Instron system may move back to the initial starting position. Container 300 may then be removed from housing 110 and the amount of medicament expelled from the syringe may be measured. To perform additional tests, membrane holder 120 may be rotated such that an unused section of membrane 140 may be concentrically loaded in the testing area. Another autoinjector may be loaded into syringe housing 510 and the testing protocol may be repeated.

It is understood that preparation of the syringe sample may include removal of the needle cap of a dose precision system syringe of interest (as described in WO 2019/118588 or WO 2020/247686), and attachment of a needle, along with its needle cover, to the exposed needle end of the syringe. The needle may then be rotated onto the syringe via a luer connection until there is resistance and the needle feels secure within the luer adaptor of the syringe. The syringe may then be filled with a sample. Alternatively, the dose precision system may be filled with a sample and then the needle cap may be removed, and a needle along with its needle cover may be attached to the exposed needle end of the syringe.

As shown in FIGS. 4A and 4B, a portion of the syringe 200 extends out of clamp 520, such that the exposed needle end extends out of the syringe and through a top first plurality of walls 112a. As shown in FIG. 4B, one of the first plurality of walls 112a includes a slit for receiving the needle. A membrane is placed into a membrane holder (not shown), which is then placed into housing 110 of fixture 100 (FIGS. 1 and 2A). Container 300 is placed into housing 110. As shown in FIG. 4B, container 300 may be placed on a portion of continuous wall 132. A program for the test system is initiated and punctures are made into membrane 140. Membrane holder 120 is rotated after each puncture for a designated number of puncture tests. A camera is set up to view the puncture and measure the depth of the syringe needle 210 as it extends into and past membrane 140.

The description above and examples are illustrative and are not intended to be restrictive. One of ordinary skill in the art may make numerous modifications and/or changes without departing from the general scope of the invention. For example, and as has been referenced, aspects of above-described embodiments may be used in any suitable combination with each other. Additionally, portions of the above-described embodiments may be removed without departing from the scope of the invention. In addition, modifications may be made to adapt a particular situation or aspect to the teachings of the various embodiments without departing from their scope. Many other embodiments will also be apparent to those of skill in the art upon reviewing the above description.

Embodiments of the present disclosure may include the following features:

Item 1. A fixture for an automatic needle injection system, the fixture comprising:
a housing including a chamber, the housing including a first plurality of walls, a second plurality of walls, and a third plurality of walls,
a holder, and
a membrane;
wherein the membrane is contained in the holder, the holder is secured within a portion of the housing, and the holder is configured to allow a portion of a needle of a syringe to pass through a portion of the holder and into the chamber of the housing.

Item 2. The fixture of item 1, wherein the fixture is configured to be secured to a bottom portion of the automatic needle injection system, wherein the syringe and fixture are coaxially aligned with the automatic needle injection system.

Item 3. The fixture of item 1, wherein the holder includes a first portion, a second portion, and a plurality of cutouts, and the membrane is secured in the holder between the first portion and the second portion.

Item 4. The fixture of item 3, wherein the first portion includes a first plurality of cutouts and the second portion includes a second plurality of cutouts, the first plurality of cutouts aligning with the second plurality of cutouts.

Item 5. The fixture of item 3, wherein the plurality of cutouts on the holder designate puncture sites for receiving the needle.

Item 6. The fixture of item 1, wherein the holder is configured to rotate within the housing.

Item 7. The fixture of item 1, wherein the membrane has a thickness ranging from about 0.1 mm to about 1.0 mm.

Item 8. The fixture of item 1, wherein the holder has a height ranging from about 1 mm to about 10 mm.

Item 9. The fixture of item 1, further comprising a container for receiving a sample from the syringe.

Item 10. The fixture of item 1, wherein the holder is a cassette and the membrane is configured to be fed through the cassette.

Item 11. A fixture for an automatic needle injection system, the fixture comprising:
a housing including a chamber,
a holder including a first portion, a second portion, and a plurality of cutouts, and
a membrane, wherein the membrane is secured between the first portion and the second portion of the holder;
wherein the holder is secured within a portion of the housing and the holder is configured to allow a portion of a needle of a syringe to pass through a portion of the holder and into the chamber of the housing.

Item 12. The fixture of item 11, wherein the holder is configured to rotate within the housing.

Item 13. The fixture of item 11, wherein the holder has a height ranging from about 1 mm to about 10 mm.

Item 14. The fixture of item 11, further comprising a container for receiving a sample from the syringe.

Item 15. The fixture of item 11, wherein the plurality of cutouts on the holder designate puncture sites for receiving the needle.

Item 16. A method for testing a syringe, the method comprising:
securing a fixture to an automatic needle injection system, the fixture comprising:
   a housing including a chamber;
   a holder; and
   a membrane;
   wherein the membrane is contained in the holder, the holder is secured within a portion of the housing, and the holder is configured to allow a needle of the syringe to pass through the holder and into the chamber of the housing, inserting a syringe into the automatic needle injection system, and programing the automatic needle injection system such that a needle of the syringe punctures the membrane.

Item 17. The method of item 16, further comprising securing the fixture to a base portion of the test system.

Item 18. The method of item 16, wherein the test system is adjusted such that an unused portion of the membrane is punctured for each test run.

Item 19. The method of item 16, wherein the needle extends through a portion of the housing, a portion of the holder, and into the chamber.

Item 20. The method of item 16, wherein the membrane is punctured at least 10 times.

What is claimed is:

1. A fixture for an automatic needle injection system, the fixture comprising:
a housing including a chamber, the housing including a first plurality of walls, a second plurality of walls, and a third plurality of walls,
a holder, and
a membrane;
wherein the membrane is contained in the holder, the holder is secured within a portion of the housing, and the holder is configured to allow a portion of a needle of a syringe to pass through a portion of the holder and into the chamber of the housing.

2. The fixture of claim 1, wherein the fixture is configured to be secured to a bottom portion of the automatic needle injection system, wherein the syringe and fixture are coaxially aligned with the automatic needle injection system.

3. The fixture of claim 1, wherein the holder includes a first portion, a second portion, and a plurality of cutouts, and the membrane is secured in the holder between the first portion and the second portion.

4. The fixture of claim 3, wherein the first portion includes a first plurality of cutouts and the second portion includes a second plurality of cutouts, the first plurality of cutouts aligning with the second plurality of cutouts.

5. The fixture of claim 3, wherein the plurality of cutouts on the holder designate puncture sites for receiving the needle.

6. The fixture of claim 1, wherein the holder is configured to rotate within the housing.

7. The fixture of claim 1, wherein the membrane has a thickness ranging from about 0.1 mm to about 1.0 mm.

8. The fixture of claim 1, wherein the holder has a height ranging from about 1 mm to about 10 mm.

9. The fixture of claim 1, further comprising a container for receiving a sample from the syringe.

10. The fixture of claim 1, wherein the holder is a cassette and the membrane is configured to be fed through the cassette.

11. A fixture for an automatic needle injection system, the fixture comprising:
a housing including a chamber,
a holder including a first portion, a second portion, and a plurality of cutouts, and
a membrane, wherein the membrane is secured between the first portion and the second portion of the holder;
wherein the holder is secured within a portion of the housing and the holder is configured to allow a portion of a needle of a syringe to pass through a portion of the holder and into the chamber of the housing.

12. The fixture of claim 11, wherein the holder is configured to rotate within the housing.

13. The fixture of claim 11, wherein the holder has a height ranging from about 1 mm to about 10 mm.

14. The fixture of claim 11, further comprising a container for receiving a sample from the syringe.

15. The fixture of claim 11, wherein the plurality of cutouts on the holder designate puncture sites for receiving the needle.

16. A method for testing a syringe, the method comprising:
securing a fixture to an automatic needle injection system, the fixture comprising:
a housing including a chamber;
a holder; and
a membrane;
wherein the membrane is contained in the holder, the holder is secured within a portion of the housing, and the holder is configured to allow a needle of the syringe to pass through the holder and into the chamber of the housing,
inserting a syringe into the automatic needle injection system, and
programing the automatic needle injection system such that a needle of the syringe punctures the membrane.

17. The method of claim 16, further comprising securing the fixture to a base portion of the test system.

18. The method of claim 16, wherein the test system is adjusted such that an unused portion of the membrane is punctured for each test run.

19. The method of claim 16, wherein the needle extends through a portion of the housing, a portion of the holder, and into the chamber.

20. The method of claim 16, wherein the membrane is punctured at least 10 times.
